(19) United States
(12) Patent Application Publication
(10) Pub. No.: US 2024/0000878 A1
(43) Pub. Date: Jan. 4, 2024

(54) METHODS FOR TREATMENT AND/OR PREVENTION OF VIRAL INFECTIONS

(71) Applicant: Xlear, Inc., Orem, UT (US)

(72) Inventor: Nathan Jones, Orem, UT (US)

(21) Appl. No.: 18/217,994

(22) Filed: Jul. 3, 2023

Related U.S. Application Data
(60) Provisional application No. 63/358,080, filed on Jul. 1, 2022.

(51) Int. Cl. A61K 36/752; A61K 9/00; A61K 9/08; A61K 47/26; A61P 31/14; A61K 33/18; A61K 31/4402

(52) U.S. Cl. CPC A61K 36/752; A61K 9/0043; A61K 9/08; A61K 47/26; A61P 31/14; A61K 33/18; A61K 31/4402

(57) ABSTRACT

Methods for treating and/or preventing viral infections. In some implementations, a composition may be provided comprising a sugar alcohol, such as xylitol, xylose, and/or erythritol. The composition may further comprise an antihistamine and/or other active anti-viral compounds, such as grapefruit seed extract, preferably in a therapeutically effective amount and/or concentration for treating a viral infection in a human having such a viral infection.

### RELATED APPLICATIONS

This application claims the benefit under 35 U.S.C. § 119(e) of U.S. Provisional Patent Application No. 63/358,080 filed Jul. 1, 2022, and titled “METHODS FOR TREATMENT AND/OR PREVENTION OF VIRAL INFECTIONS,” which application is incorporated herein by reference in its entirety.

### SUMMARY

Disclosed herein are examples of implementations of methods for treatment and/or prevention of viral infections. In some implementations, viral infections, including infections of strains of SARS-CoV-2, RSV, H1N1, and other viruses, may be prevented and/or treated by administering compositions into a subject's nasal cavity, sinuses, and/or tracheal/bronchial passages.

The present inventor has discovered that certain ingredients, such as xylitol or, in some cases, one or more other polysaccharides, may be used to block or at least inhibit a virus from adhering to tissue, such as tissue in the nasal passages/cavities and/or other regions of the airway, such as the pharynx, larynx, trachea, and/or bronchi. Another ingredient, such as a virucidal agent, may also be part of the composition and/or treatment regimen in order to kill the virus. Research into viral adhesion blocking proteins and other therapeutics is ongoing throughout the world, but the present inventor has discovered that the compositions disclosed herein, including sugar alcohols, for example, can effectively block viral adhesion without the costly development of new pharmaceutical formulations. Adding virucidal elements to the composition may further enhance the ability of the composition to prevent and/or treat viral infections.

In some implementations, a nasal solution for treating, alleviating, or preventing a viral infection may therefore be administered to a patient and/or subject. The solution may be administered, for example, preventively in an environment known or suspected to have virus present, and may thereby decrease the likelihood of an infection by, for example, decreasing the viral load in the nasal mucosa of the subject, such as in the nasal cavity, sinuses, and/or tracheal/bronchial passages.

In some implementations, the solution may comprise an active ingredient comprising a sugar alcohol, in some cases in combination with an active ingredient comprising a virucidal agent, such as iodine and/or grapefruit seed extract. In some such implementations, the sugar alcohol may comprise at least one of xylitol, xylose, erythritol, erythrose, ribose, ketose, and/or arabinose. In some such implementations, the sugar alcohol may comprise xylitol and/or erythritol. In some implementations, other non-hexose polysaccharides may be included.

In some implementations, the solution may comprise, either additionally or alternatively, another active ingredient, such as grapefruit seed extract and/or an antihistamine, such as chlorpheniramine maleate. In some implementations, other antihistamines may be used, such as brompheniramine, cetirizine, clemastine, diphenhydramine, fexofenadine, and/or loratadine. In some cases, the additional active ingredient may comprise an additional virucidal agent.

In some implementations, the composition may comprise a nasal solution. In such implementations, the nasal solution may comprise a nasal spray, and may further comprise use of a nasal spray bottle configured to deliver the nasal solution. Alternatively, the nasal solution may comprise a nasal dropper configured to deliver the nasal solution in a liquid drop form. In other embodiments, the nasal solution may comprise a gel.

In some implementations, the method may comprise identifying a subject having an infection and providing a solution. The solution may comprise a composition including one or more of the active ingredients described herein in an amount and/or concentration effective for treating the infection. For example, the solution may comprise one or more of the active, antiviral agents disclosed herein, preferably in a concentration of at least 3%, and more preferably at least 5% for the sugar alcohol antiviral agents.

For embodiments and implementations comprising grapefruit seed extract, this may be present in a concentration of at least 0.1%, and more preferably at least 0.2%. For the antiviral agents disclosed herein that are also antihistamines, this agent may be present in the composition in a concentration of at least 0.5%, and more preferably at least 1%. Such methods may further comprise delivering the solution into at least one of the subject's nose, eyes, mouth, and throat. Unless otherwise noted, the concentrations provided herein are to be considered measured in weight by volume concentrations.

In some implementations, the solution may comprise a nasal solution, and, in such implementations, the step of delivering the solution may comprise delivering the nasal solution into the subject's nose.

In a more specific example of a method for treatment of a viral infection using a nasal solution, the method may comprise identifying a subject having a viral infection, such as an infection of a H1N1, RSV, or SARS-CoV-2 virus. A dose of a nasal solution may then be delivered into the subject's nasal passage. The nasal solution may comprise xylitol and/or xylose in a concentration of at least 3% or at least about 3%. In some cases, the concentration may be higher, such as at least 5% or at least about 5%. In some embodiments and implementations, the concentration may comprise xylitol, xylose, and/or another non-hexose sugar alcohol in a concentration of between about 1% and about 15%.

In some implementations, the xylitol and/or xylose may be present in the nasal solution in a therapeutically effective concentration for treating the viral infection, such as a concentration of at least 3% or at least about 3%, or more preferably at least 5% or at least about 5%. However, in some implementations and embodiments, xylitol, xylose, erythritol, and/or another non-hexose sugar alcohol may be present in the nasal solution in a range of between about 1% and about 15%.

In some implementations, the viral infection may comprise a coronavirus infection, such as a SARS-CoV-2 infection.

In some implementations, the nasal solution may further comprise a virucidal agent, such as iodine and/or grapefruit seed extract. In some such implementations, the virucidal agent may be present in a concentration of at least 0.2%, or at least about 0.2%.

In some implementations, the dose may be part of a treatment regimen, such as a treatment regimen comprising delivering the nasal solution in each nostril of the subject at least three times daily.

In another specific example of a method for treatment of a viral infection using a nasal solution, the method may comprise identifying a subject having a viral infection, such as a viral infection selected from the group consisting of H1N1, RSV, and SARS-CoV-2. A dose of a nasal solution may then be delivered into the subject's nasal passage. The nasal solution may comprise a sugar alcohol, such as xylitol, xylose, and/or erythritol, preferably in a concentration of at least 3%, or more preferably at least 5%.

In some implementations, the nasal solution may further comprise a virucidal agent, such as grapefruit seed extract.

In an example of a method for prevention of a viral infection by decreasing a viral load in nasal mucosa, the nasal cavity, sinuses, and/or tracheal/bronchial passages using a nasal solution, the method may comprise identifying a subject either having a nasal viral load or being in an environment known or suspected to have a virus present, such as a crowded room during a pandemic, a hospital, or a room with a person who has tested positive for a viral infection, for example. A dose of a nasal solution may then be delivered into the subject's nasal passage as a preventative measure. The nasal solution may comprise a sugar alcohol, such as xylitol, xylose, and/or erythritol, or another preferably non-hexose sugar alcohol, preferably in a concentration of at least 3%, or more preferably at least 5%.

In some implementations, the virus in the environment and/or the virus making up the nasal viral load may be H1N1, RSV, SARS-CoV-2, or another coronavirus.

In some implementations, the method may further comprise delivering a dose of the nasal solution into each nostril of the subject daily, or in some cases multiple times daily (such as three or more times daily) until the nasal viral load has been confirmed to have been reduced to a subclinical level, until the subject has been removed from the environment, or until the environment has been confirmed to no longer have the virus present.

In some implementations, the nasal solution may further comprise a virucidal agent, such as iodine and/or grapefruit seed extract, which virucidal agent may be present in some implementations in a concentration of at least 0.1% or at least about 0.1%, or more preferably at least 0.2% or at least about 0.2%.

The features, structures, steps, or characteristics disclosed herein in connection with one embodiment may be combined in any suitable manner in one or more alternative embodiments.

### DETAILED DESCRIPTION

In various implementations disclosed herein, compositions including, for example, xylitol, other sugar alcohols, such as erythritol and/or other active ingredients, such as chlorpheniramine maleate and/or grapefruit seed extract, may be administered to a subject to treat and/or prevent a viral infection. In some such implementations, the subject may receive the composition by way of a nasal spray, which may allow the composition to enter various regions of the airways, including not only the nasal passages and cavities, but in some cases the pharynx, larynx, trachea, and/or bronchi, where certain types of viruses, particularly respiratory viruses, are often found.

In some implementations in which the active ingredient is a sugar alcohol, the sugar alcohol may be present in the composition in a concentration of between about 3% and about 13% weight by volume. In some such implementations, the sugar alcohol may be present in the composition in a concentration of between about 4% and about 6% weight by volume. In some such implementations, the sugar alcohol may be present in the composition in a concentration of about 5% weight by volume.

In some implementations in which the active ingredient is grapefruit seed extract, the grapefruit seed extract may be present in the composition in a concentration of between about 0.1% and about 0.4% weight by volume. In some such implementations, the grapefruit seed extract may be present in the composition in a concentration of about 0.2% weight by volume.

In some implementations in which the active ingredient is an antihistamine, such as chlorpheniramine maleate, the antihistamine may be present in the composition in a concentration of between about 0.01% and about 2% weight by volume. In some such implementations, the chlorpheniramine maleate or other antihistamine may be present in the composition in a concentration of between about 0.2% and about 1.5% weight by volume. In some such implementations, the chlorpheniramine maleate or other antihistamine may be present in the composition in a concentration of about 1% weight by volume.

### Example 1

In a particular example of a model treatment protocol used to validate various compositions and treatment methodologies and regimens disclosed herein, the antiviral activity of five different active compounds were evaluated against Influenza A/CA/07/09 (H1N1), Respiratory Syncytial Virus (RSV) strain A2, and SARS-CoV-2 strain USA/PHC658/2021 (B.1.617.2; delta) in a highly differentiated, three-dimensional (3-D), in vitro model of normal, human-derived tracheal/bronchial epithelial (TBE) cells. The compounds were tested at various concentrations indicated in Tables 1-3 below in duplicate inserts of the 3D tissue models of the human airway (MatTek Life Sciences). Antiviral activity was measured by virus yield reduction assays on day 3 (H1N1), day 5 (RSV), or day 6 (SARSC-V2) after infection.

The compounds obtained as solids were dissolved in the MatTek culture medium (AIR-100-MM) and further diluted to the test dilutions. Other compounds, such as sorbitol (45%) and GSE (43%) were received in solution and were further diluted to 5% test dilutions in the culture medium. Ribavirin (ICN Pharmaceuticals, Inc. Costa Mesa, CA) or Remdesivir (MedChemExpress, cat #HY-104077) were used during testing as positive controls.

The EpiAirway™ model was used during testing, which consists of normal, human-derived tracheal/bronchial epithelial (TBE) cells that have been cultured to form a multi-layered, highly differentiated model which closely resembles the epithelial tissue of the respiratory tract. Thus, it is anticipated that the test results may be used to simulate treatment of human patients, or prevention of infection by viruses in human subjects, by application of the various compounds, in some cases in an aqueous solution including water and/or other pharmaceutically acceptable carrier components to facilitate delivery into a subject's nasal and/or upper airway passages.

The cell cultures were made to order by MatTek Life Sciences (https://www.mattek.com) (Ashland, MA) and arrived in kits with either 12- or 24-well inserts each. The TBE cells were grown on 6 mm mesh disks in transwell inserts. During transportation the tissues were stabilized on a sheet of agarose, which was removed upon receipt. One insert was estimated to consist of approximately 1.2×106 cells. Kits of cell inserts (EpiAirway™ AIR-100, AIR-112) originated from a single, healthy, non-smoker donor #9831. Upon arrival, the cell transwell inserts were immediately transferred to individual wells of a 6-well plate according to manufacturer's instructions, and 1 mL of MatTek's proprietary culture medium (AIR-100-MM) was added to the basolateral side, whereas the apical side was exposed to a humidified 5% CO2 environment.

The TBE cells were cultured at 37° C. for a minimum of one day before the start of the experiment. After the equilibration period, the mucin layer, secreted from the apical side of the cells, was removed by washing with 400 μL prewarmed 30 mM HEPES buffered saline solution 3×. Culture medium was replenished to the basal side following the wash steps. The tissues were then allowed to rest in a 37° C. and 5% CO2 environment for a minimum of 1 hour prior to the assay.

The virus stocks used in the examples were diluted in AIR-100-MM and infected at MOI 0.01 (H1N1), MOI 0.01 (RSV) and MOI 0.02 (SARS-CoV-2) CCID₅₀ per cell, respectively.

Each treatment compound (140 μL) was applied to the apical side, and culture medium was only applied to the basal side (1 mL) for a 2-hour incubation. Virus was then added (140 μL) to the apical side for a 2-hour infection period. As a virus control, some of the cells were treated with placebo (cell culture medium only). Following the infection, the apical medium was removed, wells were washed once with media, and fresh test compound was added to the apical side. The basal side was replaced with fresh medium. The cells were maintained at the air-liquid interface.

On days 3 (H1N1), 5 (RSV), or 6 (SARS-CoV-2) post-infection, the medium was removed and discarded from the basal side. Virus released into the apical compartment of the TBE cells was harvested by the addition of 400 μL of culture medium that was pre-warmed at 37° C. The contents were incubated for 30 min, mixed well, collected, thoroughly vortexed and plated on MDCK (H1N1), MA-104 cells (RSV), or Vero E6 cells (SARS-CoV-2) for VYR titration. Triplicate wells were used for virus controls.

Determination of virus titers from each treated cell culture was performed as follows. MDCK (H1N1), MA-104 cells (RSV), or Vero E6 cells (SARS-CoV-2) cells were seeded in 96-well plates and grown overnight at about 37° C. to confluence. Samples containing virus were then diluted in 10-fold increments in infection medium and 200 μL of each dilution transferred into respective wells of a 96-well microtiter plate. Four microwells were used for each dilution to determine 50% viral endpoints. After 3-7 days of incubation, each well was scored positive for virus if any cytopathic effect (CPE) was observed as compared with the uninfected control. The virus dose that was able to infect 50% of the cell cultures (CCID₅₀ per 0.2 mL) was calculated by the Reed-Muench method (1948). The resulting VYR data and log reduction values (LRV) are summarized in Tables 1-3 below. LRV is defined as the mean reduction of virus compared to the mean virus control after infection.

The first table below, Table 1, provides a summary of the antiviral efficacy of the various compounds against Influenza A/CA/07/09 (H1N1).

TABLE 1

| | | Log₁₀ | |
|---|---|---|---|
| | | CCID₅₀ | |
| | Concentration | virus per | |
| Test Compounds | (%) | 0.2 mL | LRV |
| Sorbitol | 5 | 6.30 | 1.47 |
| | 5 | 5.30 | |
| Erythritol | 5 | 3.50 | 3.17 |
| | 5 | 4.67 | |
| Xylitol | 5 | 6.00 | 1.27 |
| | 5 | 6.00 | |
| Grapefruit Seed | 0.2 | 1.50 | 5.77 |
| Extract | 0.2 | 1.50 | |
| Chlorpheniramine | 1 | 1.50 | 6.02 |
| Maleate | 1 | 1.00 | |
| Ribavirin | 100 μg/ml | 0.67 | EC₉₀ |
| | 10 | 5.50 | 3.1 |
| | 1 | 7.30 | SI >32 |
| Virus Control | MOI 0.1 | 7.30 | Avg. |
| Influenza | | 7.00 | 7.27 |
| A/CA/07/09 (H1N1) | | 7.50 | |

Note that in the results of Table 1, each well was scored positive for virus if any cytopathogenic effect was observed as compared with the uninfected control. In addition, the figures under the third column of the table are indicative of titer results from the virus yield reduction assay. The fourth column of the table (LRV) is the log reduction value and indicates the average reduction of virus compared to the average virus control. EC₉₀ in the table indicates a 90% effective concentration (reduce virus yield by 1 log₁₀), as determined by regression analysis.

The second table below, Table 2, provides a summary of the antiviral efficacy of the various compounds against Respiratory Syncytial Virus (RSV) strain A2.

TABLE 2

| | | Log₁₀ | |
|---|---|---|---|
| | | CCID₅₀ | |
| | Concentration | virus per | |
| Test Compounds | (%) | 0.2 mL | LRV |
| Sorbitol | 5 | 2.00 | 2.49 |
| | 5 | 2.00 | |
| Erythritol | 5 | 3.00 | 1.84 |
| | 5 | 2.30 | |
| Xylitol | 5 | 2.00 | 2.65 |
| | 5 | 1.67 | |
| Grapefruit Seed | 0.2 | 2.00 | 2.34 |
| Extract | 0.2 | 2.30 | |
| Chlorpheniramine | 1 | 1.50 | 2.99 |
| Maleate | 1 | 1.50 | |
| Ribavirin | 100 μg/ml | 1.30 | EC₉₀ |
| | 10 | 3.30 | 4.6 |
| | 1 | 4.67 | SI >22 |
| Virus Control | MOI 0.1 | 4.50 | Avg. |
| RSV A2 | | 4.67 | 4.49 |
| ATCC VR-1540 | | 4.30 | |

As with Table 1, in the results of Table 2, each well was scored positive for virus if any cytopathogenic effect was observed as compared with the uninfected control. In addition, the figures under the third column of the table are indicative of titer results from the virus yield reduction assay. The fourth column of the table (LRV) is the log reduction value and indicates the average reduction of virus compared to the average virus control. EC₉₀ in the table indicates a 90% effective concentration (reduce virus yield by 1 log₁₀), as determined by regression analysis.

The third table below, Table 3, provides a summary of the antiviral efficacy of the various compounds against SARS-CoV-2 strain USA/PHC658/2021 (B.1.617.2; delta).

TABLE 3

| | | Log₁₀ | |
|---|---|---|---|
| | | CCID₅₀ | |
| | Concentration | virus per | |
| Test Compounds | (%) | 0.2 mL | LRV |
| Sorbitol | 5 | 1.50 | 3.5 |
| | 5 | 1.67 | |
| Erythritol | 5 | 1.67 | 3.29 |
| | 5 | 2.00 | |
| Xylitol | 5 | 1.50 | 3.84 |
| | 5 | 1.00 | |
| Grapefruit Seed | 0.2 | 1.50 | 3.59 |
| Extract | 0.2 | 1.50 | |
| Chlorpheniramine | 1 | 2.30 | 2.69 |
| Maleate | 1 | 2.50 | |
| Remdesivir | 5 μg/ml | 0.67 | EC₉₀ |
| | 0.5 | 2.50 | 0.12 |
| | 0.05 | 5.00 | SI >42 |
| Virus Control | MOI 0.2 | 5.30 | Avg. |
| SARS-COV-2 | | 5.30 | 5.09 |
| USA/PHC658/2021 | | 4.67 | |
| (B.1.617.2; delta) | | | |

Again, in the results of Table 3, each well was scored positive for virus if any cytopathogenic effect was observed as compared with the uninfected control. In addition, the figures under the third column of the table are indicative of titer results from the virus yield reduction assay. The fourth column of the table (LRV) is the log reduction value and indicates the average reduction of virus compared to the average virus control. EC₉₀ in the table indicates a 90% effective concentration (reduce virus yield by 1 log₁₀), as determined by regression analysis.

As shown in all of these examples, all three of the tested sugar alcohols displayed substantial virucidal activity against the tested respiratory viruses. For example, erythritol showed antiviral activity against H1N1, sorbitol and xylitol against RSV, and all three sugar alcohols displayed antiviral activity against SARS-CoV-2.

Preliminary data further suggests that xylitol displays antiretroviral activity and/or viral blocking properties. Without being limited by theory, it is thought that, mechanistically, D-xylose is the initiating element for sulfated glycosaminoglycans (GAG) that attach to the core protein of the virus. D-xylose can be derived from xylitol, and therefore xylitol may be used as an active ingredient in a treatment composition and/or regimen, by d-xylose reductase action replenishing this carbohydrate that is targeted by the SARS-CoV-2 virus. If the virus attaches to the d-xylose position on the GAG, such as heparin sulfate, the virus can then contact the ACE2 receptor.

Additionally, again, without being limited by theory, it is thought that the virus adsorption is impaired due to D-xylose (xylitol) production of glycosaminoglycans decoy targets and hence may be blocking viral adsorption.

These results suggest that intranasally administered solutions containing xylitol and/or other sugar alcohols may be beneficial for decreasing nasal viral loads and thereby preventing viral infections, treating symptoms of viral infections, such as SARS-CoV-2 infections and/or other coronavirus infections. Some symptoms that may be treated using the compositions disclosed herein include SARS-CoV-2-induced persistent anosmia. Some implementations may result in lessening the severity and/or length of viral infections, including but not limited to SARS-CoV-2 infections or other coronavirus infections.

In preferred implementations, the compositions used in the methods disclosed herein may be sold over the counter. In addition, the use of xylitol and/or other preferably non-hexose sugar alcohols may, in addition to treating viral infections, have the benefit of treating and/or preventing bacterial infections.

It should also be noted that xylitol has also been shown to be very effective in moisturizing nasal passages and the like. Without being limited by theory, this is thought to occur because xylitol can create a hyper-osmotic solution that pulls moisture towards it from surrounding tissues without generated mucous. Thus, the combination of xylitol, or other similar sugar alcohols disclosed herein, may result in a decrease in mucous production even when used with drying antihistamines without the accompanying dryness, or at least with reduced dryness, that typically accompanies antihistamines. By combining this with the accompanying anti-bacterial, anti-viral, and other health benefits associated with xylitol and other similar sugar alcohols, a synergistic composition is provided that may be useful in replacing a series of other pharmacological agents.

The present inventor has discovered that these unique characteristics make xylitol, and possibly other related non-hexose sugar alcohols, extremely useful in disrupting bacterial biofilms. Again, without being limited by theory, it is thought that this may be due to the inability of these substances to be treated as a source of energy and taken in by bacteria (due to xylitol's similar shape to other sugars that may serve as an energy source), which leaves no room for six-carbon sugars and thereby impedes bacterial growth and reproduction by, in essence, starvation in the presence of xylitol.

In some embodiments and implementations, viral infection treatment compositions disclosed herein may comprise methods, agents, compositions, etc. disclosed in U.S. Pat. Nos. 6,054,143 and 6,258,372, both titled “XYLITOL NOSE SPRAY” and U.S. Pat. No. 6,599,883 titled “NASAL DELIVERY OF XYLITOL,” each of which is incorporated herein by reference in its entirety.

In some embodiments and implementations, administering antihistamines in conjunction with xylitol may reduce dryness associated with the use of the antihistamine compositions in an individual's nasopharynx, nose, and/or throat that would normally otherwise be associated with use of the antihistamine composition alone. In some embodiments and implementations, polysaccharides, monosaccharides and/or sugar alcohol compositions utilized in the anti-mucosal compositions disclosed herein may be prepared, at least in part, utilizing methods disclosed in U.S. Pat. Nos. 6,054,143 and 6,258,372, both titled “XYLITOL NOSE SPRAY” and U.S. Pat. No. 6,599,883 titled “NASAL DELIVERY OF XYLITOL,” each of which was previously incorporated by reference in its entirety.

The compositions disclosed herein may be delivered via an intranasal pathway. For example, in some implementations, the composition may be delivered to an individual's nasopharnyx via a nasal spray.

Anatomically, the nasopharynx is a point at which the nasal passages merge into one. It is also where the floor of the nose bends downward with the superior-posterior surface of the palate. The openings of the auditory tubes (i.e., eustachian tubes) and the posterior nasal apertures (i.e., choanae) are located within the nasopharynx. The oropharynx is located inferior to the nasopharynx and is behind the mouth. By virtue of the anatomic locations of the eustachian tube openings in the nasopharynx, nasal administration of a solution or other composition may result in a more effective exposure of the eustachian tube openings versus administration via an oral route. Accordingly, administering the composition via an intranasal pathway may be more effective than other routes of administration (e.g., orally, topically). However, it is anticipated that in other embodiments and implementations, one or more of the compositions disclosed herein may alternatively be administered orally by way of drops, a spray, a gel, a solution, or the like.

In certain implementations, administering the composition via an intranasal pathway may be performed utilizing a nasal spray bottle. The nasal spray bottle may any suitable bottle configured to retain compositions including one or more of the virucidal active ingredients disclosed herein, such as xylitol, other sugar alcohols, such as erythritol, chlorpheniramine maleate or another antihistamine, and/or grapefruit seed extract, and to distribute (e.g., spray) the composition into an individual's intranasal pathway and/or nasopharynx using a pump mechanism. Alternatively, the composition may be delivered from other nasal spray bottles by simply squeezing the bottle. In certain embodiments, the composition may be stored in the nasal spray bottle in liquid or powder form and may be distributed into the intranasal pathway and/or nasopharynx as an aerosol.

In some implementations, the composition may be administered using a bathing delivery method. A bathing delivery method may utilize a solution comprising the composition including one or more of the disclosed virucidal active ingredients contained within a dilute (e.g., a less viscous, more fluid composition), pharmaceutically acceptable carrier suitable for nasal administration.

For example, the composition may be contained within an aqueous solution including water and/or other pharmaceutically acceptable carrier components. In certain embodiments and implementations, the composition may comprise approximately 0.1% saturation of a suitable aqueous solution. In certain embodiments and implementations, the composition may comprise approximately 1-15% of a suitable aqueous solution.

The bathing delivery method may directly deliver the composition to the nasopharynx in conjunction with subsequent bathing of the nasopharyngal area. As discussed above, this may be achieved using a nasal spray bottle. However, in alternative implementations, the composition may be delivered using alternative delivery mechanisms, such as droppers, misters, atomizers, brushes, swabs, etc. In some implementations, utilizing a free-flowing, low viscosity aqueous solution may allow for a rapid and concentrated application of the composition.

In further embodiments and implementations, the composition may be administered using an adhesion delivery method. An adhesion delivery method may utilize a more viscous solution, such as a gel, to deliver the composition. In certain embodiments and implementations, the viscous solution may include a bio-adhesive agent. An adhesion delivery method may rely on the adhesion of a solution containing the composition to the nasal mucous membrane and a slow migration of the solution to the nasopharyngeal area. Utilizing a more viscous solution may provide for a more gradual and steady application of the composition to desired areas, such as within the nasal cavity, and may also increase the duration of the positive benefits of the composition, such as by decreasing the rate at which the composition is removed from the desired areas.

In certain embodiments and implementations, a solution including the composition may include a buffer, a thickening agent, a bio-adhesive, and/or a humectant. A pharmaceutically acceptable surfactant and a preservative may also be included along with one or more excipients suitable for a pharmaceutical composition.

In embodiments and implementations including a buffer, the buffer may be configured to maintain a pH level of the solution. Exemplary suitable buffers include acetate, citrate, and phosphate buffers. The thickening agent may include, for example, one or more of methylcellulose, xanthan gum, carboxyl methylcellulose, polyvinyl alcohol, hydroxpropyl cellulose, carbomer, starches, chitosans, acrylates, and mixtures thereof. In certain embodiments, these substances may also act as suitable bio-adhesives. Suitable exemplary humectants include sorbitol, propylene glycol, glycerol, and/or any combination thereof. Suitable surfactants may be anionic, cationic, or nonionic, and may include polyoxyethylene derivatives, fatty acids, and/or partial esters of sorbitol anhydrides. For example, the surfactant may include sodium lauryl sulfate, polysorbate 80, polyoxyl Stearate, polyoxy ethylene 50, fusieates, bile salts, and octoxynol. However, it should also be understood that many embodiments and implementations of the compositions disclosed herein will not need to include a buffer.

In preferred implementations of methods for treating or preventing virus infections, the composition may be administered into the nasal passage of a subject, such as by way of a nasal spray or other suitable applicator, at a frequency rate of one or two sprays in each nostril from once daily to four times daily. In a most preferred implementation, the treatment regimen may comprise administration of the composition into the nasal passage of a subject at a frequency rate of one or two sprays in each nostril three times daily, in some such implementations until the bottle is empty.

The preferred dosage range used in connection with one or more the embodiments and/or implementations disclosed herein may be from about 0.1 mL to about 0.6 mL of fluid per dose.

It will be understood by those having skill in the art that changes may be made to the details of the above-described embodiments without departing from the underlying principles presented herein. For example, the anti-viral compositions disclosed herein may be administered via liquid drops from a dropper, topically (in some cases using a cotton swab or the like), orally, via a mister or atomizer, and/or via any other suitable manner of administration. In addition, any suitable combination of various embodiments, or the ingredients, methods, or features thereof, is contemplated.

Any methods disclosed herein may comprise one or more steps or actions for performing the described method. The method steps and/or actions may be interchanged with one another. In other words, unless a specific order of steps or actions is required for proper operation of the embodiment, the order and/or use of specific steps and/or actions may be modified. It should also be understood that some implementations can be practiced without some or all of the steps disclosed herein. In addition, the steps of a method do not necessarily need to be executed in any specific order, or even sequentially, nor need the steps be executed only once, unless otherwise specified.

Throughout this specification, any reference to “one embodiment/implementation,” “an embodiment/implementation,” or “the embodiment/implementation” means that a particular feature, structure, or characteristic described in connection with that embodiment/implementation is included in at least one embodiment/implementation. Thus, the quoted phrases, or variations thereof, as recited throughout this specification are not necessarily all referring to the same embodiment/implementation.

Similarly, it should be appreciated that in the above description of embodiments, various features are sometimes grouped together in a single embodiment, FIGURE, or description thereof for the purpose of streamlining the disclosure. This method of disclosure, however, is not to be interpreted as reflecting an intention that any claim require more features than those expressly recited in that claim. Rather, inventive aspects lie in a combination of fewer than all features of any single foregoing disclosed embodiment. It will be apparent to those having skill in the art that changes may be made to the details of the above-described embodiments without departing from the underlying principles set forth herein. The scope of the present inventions should, therefore, be determined only by the following claims.

1. A method for treatment of a viral infection using a nasal solution, wherein the method comprises the steps of:
identifying a subject having a viral infection; and
delivering a dose of a nasal solution into the subject's nasal passage, wherein the nasal solution comprises xylitol and/or xylose in a concentration of at least about 3%.

2. The method of claim 1, wherein the xylitol and/or xylose is present in the nasal solution in a therapeutically effective concentration for treating the viral infection.

3. The method of claim 1, wherein the viral infection comprises a coronavirus infection.

4. The method of claim 3, wherein the viral infection comprises a SARS-CoV-2 infection.

5. The method of claim 1, wherein the xylitol and/or xylose is present in the nasal solution in a concentration of at least about 5%.

6. The method of claim 1, wherein the nasal solution further comprises a virucidal agent.

7. The method of claim 6, wherein the virucidal agent comprises one or both of iodine and grapefruit seed extract.

8. The method of claim 7, wherein the virucidal agent comprises grapefruit seed extract in a concentration of at least about 0.2%.

9. The method of claim 1, wherein the dose is part of a treatment regimen, and wherein the treatment regimen comprises delivering the nasal solution in each nostril of the subject at least three times daily.

10. The method of claim 1, wherein the viral infection comprises an infection of a respiratory syncytial virus.

11. A method for treatment of a viral infection using a nasal solution, wherein the method comprises the steps of:
identifying a subject having a viral infection, wherein the viral infection is selected from the group consisting of H1N1, RSV, and SARS-CoV-2; and
delivering a dose of a nasal solution into the subject's nasal passage, wherein the nasal solution comprises xylitol, xylose, and/or erythritol in a concentration of at least 3%.

12. The method of claim 11, wherein nasal solution comprises xylitol.

13. The method of claim 11, wherein nasal solution comprises xylitol, xylose, and/or erythritol in a concentration of at least 5%.

14. The method of claim 11, wherein nasal solution further comprises grapefruit seed extract.

15. A method for prevention of a viral infection by decreasing a viral load using a nasal solution, wherein the method comprises the steps of:
identifying a subject either having a nasal viral load or being in an environment known or suspected to have a virus present; and
delivering a dose of a nasal solution into the subject's nasal passage, wherein the nasal solution comprises xylitol, xylose, and/or erythritol in a concentration of at least 5%.

16. The method of claim 15, wherein the virus in the environment and/or a virus making up the nasal viral load is selected from the group consisting of H1N1, RSV, and SARS-CoV-2.

17. The method of claim 16, wherein the method further comprises delivering a dose of the nasal solution into each nostril of the subject daily until the nasal viral load has been confirmed to have been reduced to a subclinical level, until the subject has been removed from the environment, or until the environment has been confirmed to no longer have the virus present.

18. The method of claim 17, wherein the method comprises delivering a dose of the nasal solution into each nostril of the subject at least three times daily until the nasal viral load has been confirmed to have been reduced to a subclinical level, until the subject has been removed from the environment, or until the environment has been confirmed to no longer have the virus present.

19. The method of claim 15, wherein the nasal solution further comprises a virucidal agent.

20. The method of claim 19, wherein the virucidal agent comprises iodine and/or grapefruit seed extract in a concentration of at least about 0.2%.
